(19) United States
(12) Patent Application Publication
(10) Pub. No.: US 2015/0164468 A1
(43) Pub. Date: Jun. 18, 2015

(54) APPARATUS AND METHOD FOR PROCESSING ECHOCARDIOGRAM USING NAVIER-STOKES EQUATION

(71) Applicant: INSTITUTE FOR BASIC SCIENCE, Daejeon (KR)

(72) Inventors: CHI YOUNG AHN, DAEJEON (KR); KI WAN JEON, DAEJEON (KR); SUNG HO KANG, DAEJEON (KR)

(21) Appl. No.: 14/538,039

(22) Filed: Nov. 11, 2014

(30) Foreign Application Priority Data
Dec. 13, 2013 (KR) 10-2013-0155880

(51) Int. Cl. A61B 8/08; G06T 17/00; A61B 8/06

(52) U.S. Cl. CPC A61B 8/0883; A61B 8/488; A61B 8/5207; A61B 8/06; G06T 17/00; G06T 2207/30104

(57) ABSTRACT

An apparatus and method for processing an echocardiogram using the Navier-Stokes equation is provided. The apparatus includes an ultrasonic sensor that transmits ultrasonic waves to the heart in three different directions and receives echoes thereof; an image processor that constructs first to third echocardiograms captured from the three different directions, respectively, based on the received echoes of the ultrasonic waves; a three-dimensional model construction unit that constructs a three-dimensional model of a left ventricle of the heart based on the first to third echocardiograms; and a blood flow vector calculation unit that calculates blood flow vectors within the left ventricle by applying the three-dimensional model of the left ventricle to boundary conditions in the Navier-Stokes equation. In echocardiogram processing, vector components of the blood flow within the left ventricle are calculated using the Navier-Stokes equation, whereby the vector components of the blood flow can be more accurately calculated.

### CROSS-REFERENCE TO RELATED APPLICATION

This application claims priority to KR 10-2013-0155880, having a filing date of Dec. 13, 2013, the entire contents of which are hereby incorporated by reference.

### FIELD OF TECHNOLOGY

The following relates to an apparatus and method for processing an echocardiogram using the Navier-Stokes equation, and more particularly, to an apparatus and method for processing an echocardiogram using the Navier-Stokes equation, which can calculate vector components of a blood flow within a left ventricle using the Navier-Stokes equation in echocardiogram processing.

### BACKGROUND

Ultrasonic image diagnostic apparatuses are widely used in medical fields to acquire information on an internal structure of a subject due to their easy mobility, noninvasive and nondestructive characteristics, and capabilities of providing images in real time.

In general, ultrasonic image diagnostic apparatuses provide two dimensional images of a subject by transmitting ultrasonic signals to the subject, receiving the ultrasonic signals reflected from the subject, followed by performing signal and image processing with respect to the reflected signals. In addition, the ultrasonic image diagnostic apparatuses also provide two dimensional color Doppler images for displaying velocity components of a blood flow in a direction of ultrasonic wave propagation by transmitting/receiving ultrasonic signals to/from fluid flowing within a subject, for example, a blood flow in blood vessels, followed by calculating Doppler frequencies.

Quantitative information on the function of the heart is required for heart disease diagnosis. The quantitative information includes left ventricular hypertrophy, stroke volume, ejection fraction, cardiac output, and the like. In recent years, a lot of attention has been paid to technologies for utilizing vorticity of a blood flow in diagnosis by calculating velocity vectors of the blood flow, followed by quantifying the vorticity of the blood flow based on the calculated velocity vectors. In order to acquire the quantitative information on the function of the heart, it is necessary to three-dimensionally identify directions and velocities of a blood flow within a left ventricle that shrinks and expands to supply blood to the whole body.

In the related art, directions and velocities of a blood flow within the heart are chiefly identified by a method of calculating blood flow vectors by administering contrast media and then tracing movement of speckles within the heart.

However, such a technique has disadvantages in that contrast media are necessarily administered to calculate information on vector components of the blood flow within the heart, and capabilities of tracing speckles of the blood flow seriously depend upon an image acquisition speed and an image quality. For this reason, it is difficult to accurately calculate three-dimensional blood flow vector information.

One example of the related art is disclosed in Korean Patent Publication No. 10-1998-0042140 (published on Aug. 17, 1998 and entitled “Ultrasonic diagnostic imaging system for analysis of left ventricular function”).

### SUMMARY

Embodiments of the present invention provide an apparatus and a method for processing an echocardiogram using the Navier-Stokes equation, in which, in echocardiogram processing, boundary conditions necessary for calculating flow vectors of fluid using the Navier-Stokes equation are obtained through echocardiograms of a left ventricle captured at three different angles, and vector components of a blood flow within the left ventricle are calculated by simulating the blood flow within the left ventricle using the Navier-Stokes equation, thereby calculating the vector components of the blood flow within the left ventricle without using contrast media.

In accordance with one aspect of embodiments of the present invention, an apparatus for processing an echocardiogram using the Navier-Stokes equation includes: an ultrasonic sensor that transmits ultrasonic waves to the heart in three different directions and receives echoes thereof; an image processor that constructs first to third echocardiograms captured from the three different directions, respectively, based on the received echoes of the ultrasonic waves; a three-dimensional model construction unit that constructs a three-dimensional model of a left ventricle of the heart based on the first to third echocardiograms; and a blood flow vector calculation unit that calculates blood flow vectors within the left ventricle by applying the three-dimensional model of the left ventricle to boundary conditions in the Navier-Stokes equation.

The first to third echocardiograms may be apical views.

The first to third echocardiograms may be second to fourth ventricle sectional views, respectively.

The three-dimensional model construction unit may construct a three-dimensional model for motion of the left ventricle by matching the first to third echocardiograms for each step of a cardiac cycle.

The blood flow vector calculation unit may correct the calculated blood flow vectors based on Doppler information on a blood flow within the heart.

In accordance with another aspect of the embodiments of the present invention, a method of processing an echocardiogram using the Navier-Stokes equation includes: constructing, by an image processor, first to third echocardiograms captured from different directions; constructing, by a three-dimensional model construction unit, a three-dimensional model of a left ventricle of the heart based on the first to third echocardiograms; and calculating, by a blood flow vector calculation unit, blood flow vectors within the left ventricle by applying the three-dimensional model of the left ventricle to boundary conditions in the Navier-Stokes equation.

Constructing the first to third echocardiograms may include constructing the first to third echocardiograms based on echoes of ultrasonic waves transmitted to the heart by an ultrasonic sensor in three different directions.

The first to third echocardiograms may be apical views.

The first to third echocardiograms may be second to fourth ventricle sectional views, respectively.

Constructing the three-dimensional model may include constructing, by the three-dimensional model construction unit, a three-dimensional model for motion of the left ventricle by matching the first to third echocardiograms for each step of a cardiac cycle.

Calculating the blood flow vectors may include correcting, by the blood flow vector calculation unit, the calculated blood flow vectors based on Doppler information on a blood flow within the heart.

According to the embodiments of the present invention, in echocardiogram processing, vector components of a blood flow within a left ventricle is calculated using the Navier-Stokes equation, whereby the vector components of the blood flow within the left ventricle can be more accurately calculated without using contrast media, thereby enhancing convenience and accuracy of cardiac diagnosis using echocardiograms.

### BRIEF DESCRIPTION

Some of the embodiments will be described in detail, with reference to the following figures, wherein like designations denote like members, wherein:

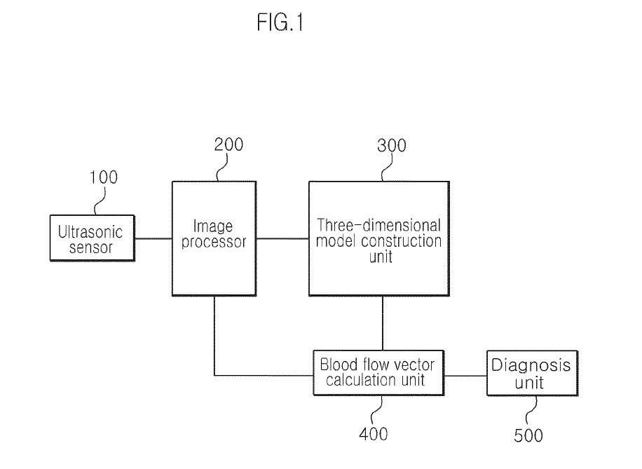

FIG. 1 is a block diagram of an apparatus for processing an echocardiogram using the Navier-Stokes equation according to one embodiment of the present invention;

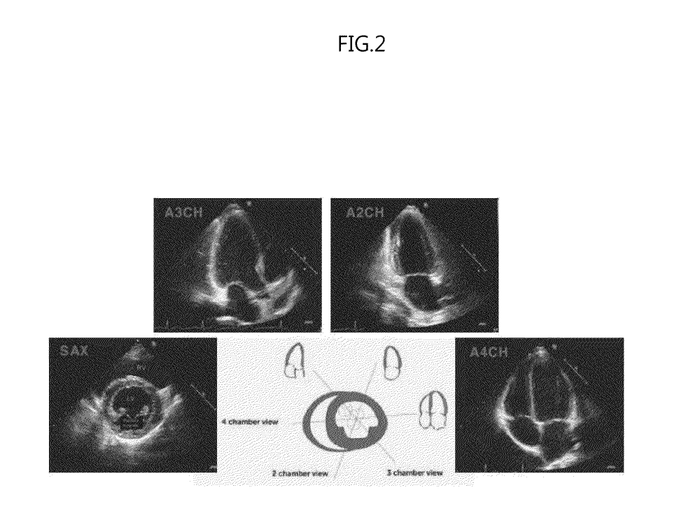

FIG. 2 shows an example of echocardiograms captured from different directions by an image processor of an embodiment of the present invention;

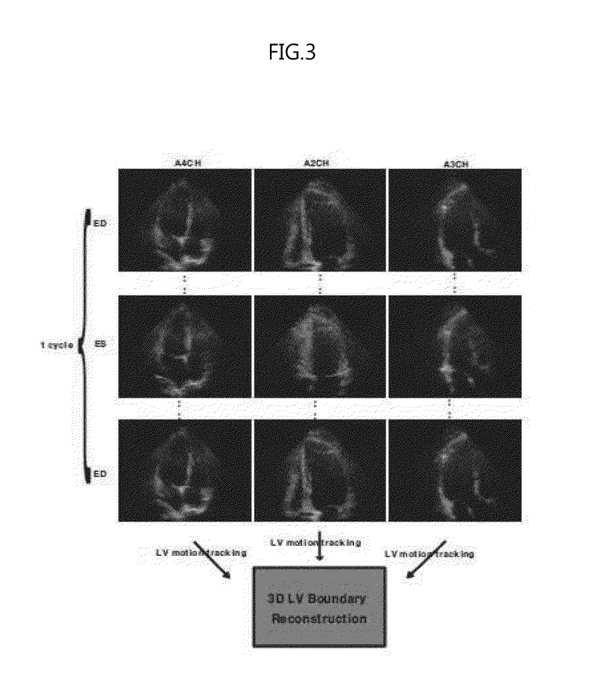

FIG. 3 shows an example of echocardiograms which a three-dimensional-model construction unit of an embodiment of the present invention matches for each step of a cardiac cycle; and

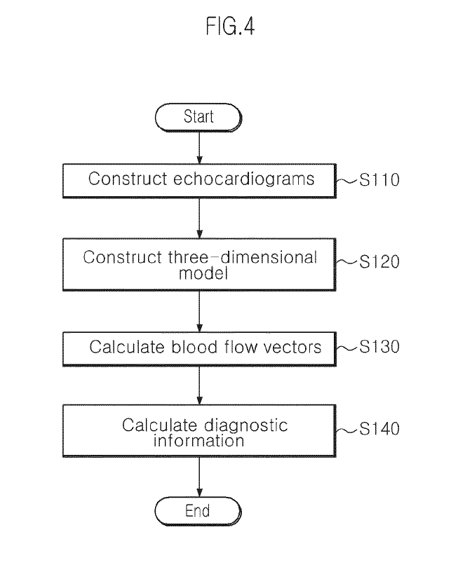

FIG. 4 is a flowchart showing a method of processing an echocardiogram using the Navier-Stokes equation according to one embodiment of the present invention.

### DETAILED DESCRIPTION

Hereinafter, an apparatus and method for processing an echocardiogram using the Navier-Stokes equation according to embodiments of the present invention will be described in detail with reference to the accompanying drawings. In describing the apparatus and the method, thicknesses of lines or sizes of components shown in the drawings may be exaggerated for clarity and convenience of description. In addition, the terms to be described below are defined in view of functions in the embodiments of the present invention and may be different according to intentions of users and operators or customs. Accordingly, the terms should be defined based on contents throughout the specification.

FIG. 1 is a block diagram of an apparatus for processing an echocardiogram using the Navier-Stokes equation according to one embodiment of the present invention.

Referring to FIG. 1, the apparatus for processing an echocardiogram using the Navier-Stokes equation according to the embodiment of the invention may include an ultrasonic sensor 100, an image processor 200, a three-dimensional model construction unit 300, and a blood flow vector calculation unit 400.

The ultrasonic sensor 100 transmits ultrasonic waves to the heart in three different directions and receives echoes thereof

In this case, the ultrasonic sensor 100 may transmit the ultrasonic waves to the heart for the same cardiac cycle in the three different directions.

Based on the echoes of the ultrasonic waves, received from the different directions by the ultrasonic sensor 100, the image processor 200 constructs first to third echocardiograms captured from the different directions.

Thus, the image processor 200 may acquire 2D sectional images of the heart captured from the three different directions for the same cardiac cycle.

In this case, the first to third echocardiograms may be apical views. The apical views are obtained by capturing internal sections of the heart along a long axis of the heart.

In this embodiment, fluid dynamic boundary conditions are deduced by constructing a three-dimensional model of the heart based on the echocardiograms, and therefore, it is desirable to obtain the apical views captured from three different directions.

Here, the first to third echocardiograms may be second to fourth ventricle sectional views, respectively.

FIG. 2 shows an example of echocardiograms captured from different directions and constructed by the image processor of the embodiments of the present invention. In FIG. 2, A2CH represents a second ventricle sectional view, A3CH represents a third ventricle sectional view, A4CH represents a fourth ventricle sectional view, and SAX represents a short axis sectional view. The short axis sectional view is obtained by capturing an internal section of the heart in a direction perpendicular to a long axis of the heart.

As shown in FIG. 2, when the heart is captured such that three apical views, captured as echocardiograms, correspond to second to fourth ventricle sectional views, respectively, the echocardiograms may be echocardiograms captured from three different directions, due to an anatomical structure of the heart. When the echocardiograms are taken from the three different directions in the aforementioned manner, anatomical boundary data for all ventricles of the heart can be obtained. Thus, a three-dimensional model of a left ventricle of the heart may be constructed based on the three echocardiograms.

The three-dimensional model construction unit 300 constructs the three-dimensional model of the left ventricle of the heart based on the first to third echocardiograms.

At this time, the three-dimensional model construction unit 300 may construct the three-dimensional model for motion of the left ventricle of the heart by matching the first to third echocardiograms for each step of a cardiac cycle.

FIG. 3 shows one example of echocardiograms which the three-dimensional model construction unit 300 of the embodiments of the present invention matches for each step of a cardiac cycle.

As shown in FIG. 3, the three-dimensional model construction unit 300 may match the first to third echocardiograms, generated based on the echoes received by the ultrasonic sensor 100, for the same step of the cardiac cycle.

One three-dimensional model may be constructed based on the first to third echocardiograms captured in the same step of the cardiac cycle. Therefore, when three-dimensional models are constructed for respective steps of the cardiac cycle based on the echocardiograms, a three-dimensional model for motion of a left ventricle according to the cardiac cycle may be constructed.

The three-dimensional model construction unit 300 may construct the three-dimensional model of the left ventricle of the heart using a level set method based on three apical views.

In the level set method, the term “zero level set” means a surface composed of a set of vectors for which a level set function has a value of 0, and the level set function has a positive value inside a region surrounded by the zero level set and a negative value outside the region, with a vector, indicating a position, as a factor.

Thus, the three-dimensional model of the left ventricle of the heart may be constructed by numerically calculating the zero level set. For example, to include left ventricle boundary data of a surface for which a zero level set is measured, energy of a level set is given by Equation 1:

E(Γ)=E(φ)=┌∫d^{p}(x)δ(φ(x))|∇φ(x)|dx┐^{1/x}

where Γ denotes a zero level set, φ denotes a level set function, x denotes a position vector, δ(x) denotes a one dimensional delta function, and δ(φ(x)|∇φ(x)|dx denotes a surface element in a zero level set of φ.

Therefore, for each step of the cardiac cycle, a three-dimensional model of the left ventricle of the heart may be constructed by Equation 1 using surface energy obtained from the echocardiograms.

The blood flow vector calculation unit 400 calculates blood flow vectors in the left ventricle of the heart by applying the three-dimensional model of the left ventricle, constructed by the three-dimensional model construction unit 300, to boundary conditions in the Navier- Stokes equation.

In this case, the blood flow vector calculation unit 400 may calculate the blood flow vectors in the left ventricle of the heart using a projection method, an immersed boundary method, a vortex-stream function method, or the like.

It has been known that movement of fluid, including vortices and eddies, can be modeled by applying boundary conditions to the Navier-Stokes equation which is a partial differential equation describing fluid dynamics.

The Navier-Stokes equation for calculating the blood flow vectors in the left ventricle is given by Equation 2:

$$\frac{d u}{d t} + u \cdot ∇ u = - ∇ + v {∇}^{2} u + f$$

where u denotes a velocity vector of fluid, p denotes pressure, v denotes coefficient of viscosity, and f denotes external force exerted on fluid.

In addition, the blood flow vector calculation unit 400 may correct the calculated blood flow vectors based on Doppler information on a blood flow within the heart.

Although a method of calculating vector components of a blood flow within blood vessels based on Doppler information obtained from echocardiograms is known in the art, the Doppler information informs of only axial velocity components of fluid. Thus, three-dimensional velocity vectors of the blood flow, including vortices occurring within a ventricle but not the blood vessels, cannot be calculated only with the Doppler information. However, the three-dimensional velocity vectors of the blood flow within the left ventricle can be more accurately calculated by correcting the three-dimensional velocity vectors, calculated using the Navier-Stokes equation, to Doppler information.

For example, a data assimilation method may be used to correct the calculated velocity vectors of the blood flow.

In addition, the apparatus for processing an echocardiogram using the Navier-Stokes equation according to the embodiment of the present invention may further include a diagnosis unit 500.

The diagnosis unit 500 calculates quantitative diagnostic information on the function of the heart based on the blood flow vectors calculated by the blood flow vector calculation unit 400.

The diagnosis unit 500 may diagnose functional disturbance of the left ventricle using quantitative diagnostic information on vorticity of the calculated blood flow vectors. The quantitative diagnostic information on the vorticity of the blood flow includes a width and a length of vortices, longitudinal and transverse positions of vortices with respect to the left ventricle, and the like.

FIG. 4 is a flowchart showing a method of processing an echocardiogram using the Navier-Stokes equation according to one embodiment of the present invention. The method of processing an echocardiogram using the Navier-Stokes equation will be described with reference to FIG. 4.

First, the image processor 200 constructs first to third echocardiograms captured from three different directions, based on echoes corresponding to ultrasonic waves transmitted to the heart by the ultrasonic sensor 100 in the three different directions (S110).

In this case, the first to third echocardiograms may be apical views.

In addition, the first to third echocardiograms may be second to fourth ventricle sectional views, respectively.

As described above with reference to FIG. 2, when the heart is captured such that the three apical views captured as echocardiograms correspond to the second to fourth ventricle sectional views, respectively, the echocardiograms may be echocardiograms captured from the three different directions, due to an anatomical structure of the heart. When the echocardiograms are taken from the three different directions in the above-described manner, anatomical boundary data for all ventricles of the heart can be obtained. Thus, a three-dimensional model of a left ventricle of the heart may be constructed based on the three echocardiograms.

Next, the three-dimensional model construction unit 300 constructs a three-dimensional model of the left ventricle of the heart, based on the first to third echocardiograms (S120).

The three-dimensional model construction unit 300 may construct the three-dimensional model for motion of the left ventricle by matching the first to third echocardiograms with respective steps of a cardiac cycle.

As described above with reference to FIG. 3, the three-dimensional model construction unit 300 may match the first to third echocardiograms, generated based on the echoes received by the ultrasonic sensor 100, for the same step of the cardiac cycle.

One three-dimensional model may be constructed based on the first to third echocardiograms captured in the same step of the cardiac cycle. Thus, when three-dimensional models are constructed for respective steps of the cardiac cycle based on the echocardiograms, a three-dimensional model for motion of the left ventricle according to the cardiac cycle may be constructed.

At this time, for each step of the cardiac cycle, a three-dimensional model of the left ventricle of the heart may be constructed by Equation 1 using surface energy obtained from the echocardiograms.

Then, the blood flow vector calculation unit 400 calculates blood flow vectors in the left ventricle of the heart by applying the three-dimensional model of the left ventricle to boundary conditions in the Navier-Stokes equation.

At this time, the blood flow vector calculation unit 400 may calculate the blood flow vectors in the left ventricle of the heart using a projection method, an immersed boundary method, a vortex-stream function method, or the like.

As described above, the Navier-Stokes equation is a partial differential equation describing fluid dynamics, and it has been known that movement of fluid, including vortices and eddies, can be modeled by applying boundary conditions to the Navier-Stokes equation.

Here, the blood flow vector calculation unit 400 may correct the calculated blood flow vectors based on Doppler information on a blood flow within the heart.

Although a method of calculating vector components of a blood flow within blood vessels based on Doppler information obtained from echocardiograms is known in the art, the Doppler information informs of only axial velocity components of fluid. Thus, three-dimensional velocity vectors of a blood flow, including vortices occurring within a ventricle but not the blood vessels, cannot be calculated only with the Doppler information. However, the three-dimensional velocity vectors of the blood flow within the left ventricle can be more accurately calculated by correcting the three-dimensional velocity vectors, calculated using the Navier-Stokes equation, to Doppler information.

In addition, a data assimilation method may be used to correct the calculated velocity vectors of the blood flow.

Then, the diagnosis unit 500 calculates quantitative diagnostic information on the function of the heart based on the blood flow vectors calculated by the blood flow vector calculation unit 400 (S140), and then terminates the process.

The diagnosis unit 500 may diagnose functional disturbance of the left ventricle using quantitative diagnostic information on vorticity of the calculated blood flow vectors. The quantitative diagnostic information on the vorticity of the blood flow includes a width and a length of vortices, longitudinal and transverse positions of vortices with respect to the left ventricle, and the like.

As such, according to this embodiment, in echocardiogram processing, the vector components of the blood flow within the left ventricle are calculated using the Navier-Stokes equation, whereby the vector components of the blood flow within the left ventricle can be more accurately calculated without using contrast media, thereby enhancing convenience and accuracy of cardiac diagnosis using echocardiograms.

Although the present invention has been described with reference to some embodiments in conjunction with the drawings, it should be understood that these embodiments are provided for illustration only and that various modifications and other equivalent embodiments can be made without departing from the spirit and the scope of the present invention. Thus, the technical scope of the present invention should be determined by the attached claims.

What is claimed is:

1. An apparatus for processing an echocardiogram using the Navier-Stokes equation, comprising:
an ultrasonic sensor that transmits ultrasonic waves to the heart in three different directions and receives echoes thereof;
an image processor that constructs first to third echocardiograms captured from the three different directions, respectively, based on the received echoes of the ultrasonic waves;
a three-dimensional model construction unit that constructs a three-dimensional model of a left ventricle of the heart based on the first to third echocardiograms; and
a blood flow vector calculation unit that calculates blood flow vectors within the left ventricle by applying the three-dimensional model of the left ventricle to boundary conditions in the Navier-Stokes equation.

2. The apparatus according to claim 1, wherein the first to third echocardiograms are apical views.

3. The apparatus according to claim 1, wherein the first to third echocardiograms are second to fourth ventricle sectional views, respectively.

4. The apparatus according to claim 1, wherein the three-dimensional model construction unit constructs a three-dimensional model for motion of the left ventricle by matching the first to third echocardiograms for each step of a cardiac cycle.

5. The apparatus according to claim 1, wherein the blood flow vector calculation unit corrects the calculated blood flow vectors based on Doppler information on a blood flow within the heart.

6. A method of processing an echocardiogram using the Navier-Stokes equation, comprising:
constructing, by an image processor, first to third echocardiograms captured from different directions;
constructing, by a three-dimensional model construction unit, a three-dimensional model of a left ventricle of the heart based on the first to third echocardiograms; and
calculating, by a blood flow vector calculation unit, blood flow vectors within the left ventricle by applying the three-dimensional model of the left ventricle to boundary conditions in the Navier-Stokes equation.

7. The method according to claim 6, wherein constructing the first to third echocardiograms comprises: constructing the first to third echocardiograms based on echoes of ultrasonic waves transmitted to the heart by an ultrasonic sensor in three different directions.

8. The method according to claim 6, wherein the first to third echocardiograms are apical views.

9. The method according to claim 6, wherein the first to third echocardiograms are second to fourth ventricle sectional views, respectively.

10. The method according to claim 6, wherein constructing the three-dimensional model comprises: constructing, by the three-dimensional model construction unit, a three-dimensional model for motion of the left ventricle by matching the first to third echocardiograms for each step of a cardiac cycle.

11. The method according to claim 6, wherein calculating the blood flow vectors comprises: correcting, by the blood flow vector calculation unit, the calculated blood flow vectors based on Doppler information on a blood flow within the heart.
